(19) United States
(12) Patent Application Publication
(10) Pub. No.: US 2022/0230720 A1
(43) Pub. Date: Jul. 21, 2022

(54) CORRECTING AN EXAMINATION REPORT

(71) Applicant: KONINKLIJKE PHILIPS N.V., EINDHOVEN (NL)

(72) Inventors: Prescott Peter KLASSEN, CAMBRIDGE, MA (US); Amir Mohammad TAHMASEBI MARAGHOOSH, ARLINGTON, MA (US); Gabriel Ryan MANKOVICH, BOSTON, MA (US); Robbert Christiaan VAN OMMERING, CAMBRIDGE (MA)

(21) Appl. No.: 17/609,550

(22) PCT Filed: May 8, 2020
(86) PCT No.: PCT/EP2020/062875

§ 371 Date: Nov. 8, 2021

Related U.S. Application Data
(60) Provisional application No. 62/848,138, filed on May 15, 2019.

(51) Int. Cl. G16H 15/00; G06V 30/41; G16H 10/60; G06F 40/30; G06F 40/174

(52) U.S. Cl. CPC G16H 15/00; G06V 30/41; G06F 40/174; G06F 40/30; G16H 10/60

(57) ABSTRACT

Methods and systems for correcting an examination report. The methods described herein extract examination and semantic data from an examination report, and identify any discrepancies between the extracted examination data and the extracted semantic data. The methods described herein then receive a resolution strategy regarding how to resolve any identified discrepancies and then resolve any identified discrepancies based on the resolution strategy.

### TECHNICAL FIELD

Embodiments described herein generally relate to systems and methods for correcting an examination report and, more particularly but not exclusively, to systems and methods for correcting an examination report based on examination data and semantic data.

### BACKGROUND

In modern healthcare departments, reporting software systems may expedite the authoring of reports by offering users such as clinicians multiple ways of recording their impressions and findings when analyzing or otherwise populating an examination document. For example, these systems may help clinicians record their impressions and findings when analyzing an image.

The list of reporting features for such products or systems is extensive and offers clinicians spelling, grammar, and controlled vocabulary support while dictating, as well as other features to expedite the transcription process and ensure a correct and accurate report. However, these tools do not address the more complex semantic or linguistic challenges involved in attempting to understand or read what the clinician or other type of user (i.e., unrelated to healthcare) is attempting to communicate.

A need exists, therefore, for methods and systems that can address the more complex challenges in correcting examination reports.

### SUMMARY

This summary is provided to introduce a selection of concepts in a simplified form that are further described below in the Detailed Description section. This summary is not intended to identify or exclude key features or essential features of the claimed subject matter, nor is it intended to be used as an aid in determining the scope of the claimed subject matter.

According to one aspect, embodiments relate to a method for correcting an examination report. The method includes extracting examination data from an examination report, extracting semantic data from the examination report, identifying a discrepancy between the extracted examination data and the extracted semantic data, receiving a resolution strategy regarding how to resolve the identified discrepancy between the extracted examination data and the extracted semantic data, and resolving the identified discrepancy between the extracted examination data and the extracted semantic data based on the resolution strategy.

In some embodiments, the examination report relates to a radiology examination.

In some embodiments, the method includes presenting, using a user interface, the identified discrepancy to a user, wherein receiving the resolution strategy includes receiving, using the user interface, user feedback regarding how to resolve the identified discrepancy between the extracted examination data and the extracted semantic data.

In some embodiments, a user provides the examination data to the examination report and includes at least one of findings, anatomies, diseases, measurements, and staging identifications related to an examination.

In some embodiments, identifying the discrepancy comprises consulting an ontology to determine whether a relationship exists between the extracted examination data and the extracted semantic data, wherein the discrepancy is identified upon determining that a relationship does not exist between the extracted examination data and the extracted semantic data.

In some embodiments, identifying the discrepancy comprises identifying the discrepancy using a neural network machine learning model trained using training examination data and training semantic data to identify relationships between extracted examination data and extracted semantic data.

In some embodiments, the extracted semantic data relates to semantic meanings of linguistic structures in the examination report.

According to another aspect, embodiments relate to a system for correcting an examination report. The system includes an interface for receiving an examination report and a processor executing instructions stored on a memory to extract examination data from an examination report, extract semantic data from the examination report, identify a discrepancy between the extracted examination data and the extracted semantic data, receive a resolution strategy regarding how to resolve the identified discrepancy between the extracted examination data and the extracted semantic data, and resolve the identified discrepancy between the extracted examination data and the extracted semantic data based on the resolution strategy.

In some embodiments, the examination report relates to a radiology examination.

In some embodiments, the system further includes a user interface for presenting the identified discrepancy to a user and receiving the resolution strategy from the user.

In some embodiments, the examination data includes at least one of findings, anatomies, diseases, measurements, and staging identifications related to an examination.

In some embodiments, the processor is further configured to identify the discrepancy by consulting an ontology to determine whether a relationship exists between the extracted examination data and the extracted semantic data, wherein the discrepancy is identified upon the processor determining that a relationship does not exist between the extracted examination data and the extracted semantic data. In some embodiments, the processor is further configured to identify the discrepancy using a neural network machine learning model using training examination data and training semantic data to identify relationships between extracted examination data and extracted semantic data.

In some embodiments, the extracted semantic data relates to semantic meanings of linguistic structures in the examination report.

According to yet another aspect, embodiments relate to a non-transitory computer-readable medium containing computer executable instructions for performing a method for correcting an examination report. The computer-readable medium includes computer-executable instructions for extracting examination data from an examination report, computer-executable instructions for extracting semantic data from the examination report, computer-executable instructions for identifying a discrepancy between the extracted examination data and the extracted semantic data, computer-executable instructions for receiving a resolution strategy regarding how to resolve the identified discrepancy between the extracted examination data and the extracted semantic data, and computer-executable instructions for resolving the identified discrepancy between the extracted examination data and the extracted semantic data based on the resolution strategy.

### BRIEF DESCRIPTION OF DRAWINGS

Non-limiting and non-exhaustive embodiments of the embodiments herein are described with reference to the following figures, wherein like reference numerals refer to like parts throughout the various views unless otherwise specified:

FIG. 1 illustrates a system for correcting an examination report in accordance with one embodiment;

FIG. 2 illustrates a workflow of the various components and data of FIG. 1 in accordance with one embodiment; and

FIG. 3 depicts a flowchart of a method for correcting an examination report in accordance with one embodiment.

### DETAILED DESCRIPTION

Various embodiments are described more fully below with reference to the accompanying drawings, which form a part hereof, and which show specific exemplary embodiments. However, the concepts of the present disclosure may be implemented in many different forms and should not be construed as limited to the embodiments set forth herein; rather, these embodiments are provided as part of a thorough and complete disclosure, to fully convey the scope of the concepts, techniques and implementations of the present disclosure to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one example implementation or technique in accordance with the present disclosure. The appearances of the phrase “in one embodiment” in various places in the specification are not necessarily all referring to the same embodiment. The appearances of the phrase “in some embodiments” in various places in the specification are not necessarily all referring to the same embodiments.

Some portions of the description that follow are presented in terms of symbolic representations of operations on non-transient signals stored within a computer memory. These descriptions and representations are used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices. Portions of the present disclosure include processes and instructions that may be embodied in software, firmware or hardware, and when embodied in software, may be downloaded to reside on and be operated from different platforms used by a variety of operating systems.

The present disclosure also relates to an apparatus for performing the operations herein. This apparatus may be specially constructed for the required purposes, or it may comprise a general-purpose computer selectively activated or reconfigured by a computer program stored in the computer or by using some cloud-based solution. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each may be coupled to a computer system bus. Furthermore, the computers referred to in the specification may include a single processor or may be architectures employing multiple processor designs for increased computing capability.

The processes and displays presented herein are not inherently related to any particular computer or other apparatus. Various general-purpose systems may also be used with programs in accordance with the teachings herein, or it may prove convenient to construct more specialized apparatus to perform one or more method steps. The structure for a variety of these systems is discussed in the description below. In addition, any particular programming language that is sufficient for achieving the techniques and implementations of the present disclosure may be used. A variety of programming languages may be used to implement the present disclosure as discussed herein.

In addition, the language used in the specification has been principally selected for readability and instructional purposes and may not have been selected to delineate or circumscribe the disclosed subject matter. Accordingly, the present disclosure is intended to be illustrative, and not limiting, of the scope of the concepts discussed herein.

Medical personnel (for simplicity, “clinicians”) frequently are tasked with reviewing imagery and other data relating to a patient's health as part of patient treatment. Clinicians often provide notes or otherwise record their findings in an examination report. In modern radiology departments, for example, reporting software systems expedite the authoring of examination reports by offering radiologists multiple ways of recording their impressions and findings when analyzing an image or other type of document related to a patient's health.

One technique involves speech-to-text technologies. In these types of techniques, a clinician may provide verbal notes while reviewing an image or report by speaking into a microphone. Speech-to-text techniques may therefore transcribe the author's verbal cues into text that is associated with the analyzed image or report.

Speech-to-text techniques are subject to errors, however. In some instances, the author may not speak clearly into the microphone, or the author may speak with an accent such that the transcription technology is unable to accurately transcribe the author's statements. In these instances, the transcribed word may be spelled correctly and grammatically appropriate. However, the transcribed word may be semantically incorrect.

Other techniques for clinicians to populate or otherwise create an examination report are mouse- and menu-driven. For example, a clinician may navigate a cursor on a screen using a mouse to select various entries from menus (e.g., drop-down menus) to add specific words from ontologies or vocabularies. Similarly, another existing technique involves the use of a keyboard to add or edit free text to a report.

These approaches frequently generate examination reports that include ambiguous references to anatomical regions of organs. In these instances, these techniques would require a human-level, co-reference resolution to understand the term's contextual meaning. For example, the term “lobe” in a report may be ambiguous as to which anatomical lobe the clinician is referring.

As another example, these existing report generation techniques may also generate reports with sentences that are opaque in meaning due to telegraphic or terse language. For example, a clinician may populate a report using brief sentences, certain terms, ellipses, etc., under the assumption that the reader will understand exactly what the clinician is intending to convey. This assumption may be correct if the clinician and eventual reader have a pre-existing relationship such that the reader can readily ascertain the clinician's intended message notwithstanding the clinician's brevity. Often times, however, the eventual reader may be unsure of the clinician's intended message.

The systems and methods described herein provide novel techniques to autonomously correct examination reports such as those in the healthcare setting. The features described herein may highlight errors and inconsistencies in the report by understanding the semantics of the words and phrases in the report and subsequently address the highlighted errors and inconsistencies.

The systems and methods described herein may rely on natural language processing, statistical machine learning, and/or neural network-based deep learning software instructions and components. The systems and methods described herein may incorporate a runtime component to seamlessly integrate with existing systems and reporting software.

The features of the systems and methods described herein therefore provide an enhancement over conventional spelling and grammar correction tools. These existing tools, for example, cannot identify or address errors based on the semantics of a report or words or phrases therein.

Although the present application is largely directed towards correcting examination reports related to radiology examinations, the features of the systems and methods herein may be incorporated into other healthcare applications. For example, the systems and methods described herein may correct any type of report related to a healthcare examination or test. The embodiments described herein are not limited to the healthcare context either.

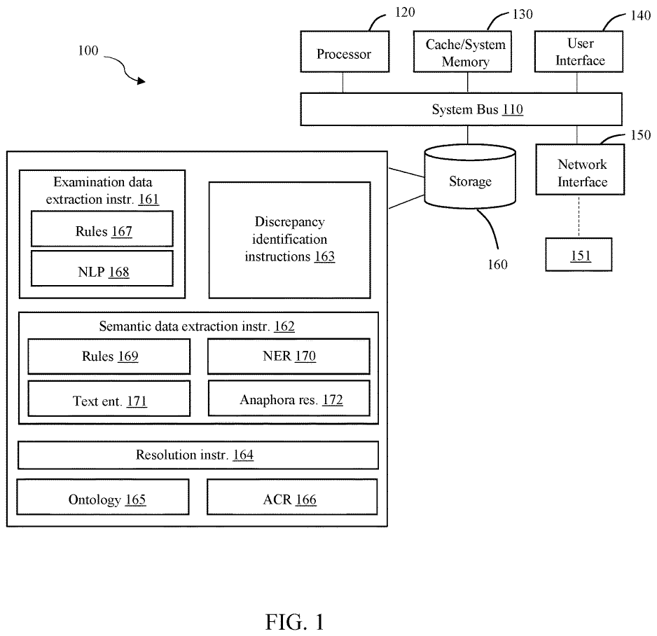

FIG. 1 illustrates a system 100 for correcting an examination report in accordance with one embodiment. The system 100 may include a processor 120, memory 130, a user interface 140, a network interface 150, and storage 160 interconnected via one or more system buses 110. It will be understood that FIG. 1 constitutes, in some respects, an abstraction and that the actual organization of the system 100 and the components thereof may differ from what is illustrated.

The processor 120 may be any hardware device capable of executing instructions stored on memory 130 or storage 160 or otherwise capable of processing data. As such, the processor 120 may include a microprocessor, field programmable gate array (FPGA), application-specific integrated circuit (ASIC), or other similar device(s).

In some embodiments, such as those relying on one or more ASICs, the functionality described as being provided in part via software may instead be configured into the design of the ASICs and, as such, the associated software may be omitted. The processor 120 may be configured as part of a user device on which the user interface 140 executes or may be located at some remote location.

The memory 130 may include various memories such as, for example L1, L2, L3 cache, or system memory. As such, the memory 130 may include static random access memory (SRAM), dynamic RAM (DRAM), flash memory, read only memory (ROM), or other similar memory devices. The exact configuration of the memory 130 may vary as long as instructions for correcting an examination report can be executed.

The user interface 140 may execute on one or more devices for enabling communication with a user such as a clinician or other type of medical personnel. For example, the user interface 140 may include a display, a microphone, a mouse, and a keyboard for receiving user commands or notes. In some embodiments, the user interface 140 may include a command line interface or graphical user interface that may be presented to a remote terminal via the network interface 150.

The user interface 140 may execute on a user device such as a PC, laptop, tablet, mobile device, smartwatch, or the like. The exact configuration of the user interface 140 and the device on which it executes may vary as long as the features of various embodiments described herein may be accomplished. The user interface 140 may enable a clinician or other type of medical personnel to view imagery related to a medical examination, input notes related to an examination, view notes related to an examination, receive instances of identified discrepancies, provide resolution instructions regarding the identified discrepancies, etc. Regardless of the exact configuration of the user interface 140, the user interface 140 may work in conjunction with any existing software or hardware to seamlessly integrate these discrepancy identification and correction techniques into the examination workflow.

The network interface 150 may include one or more devices for enabling communication with other hardware devices. For example, the network interface 150 may include a network interface card (MC) configured to communicate according to the Ethernet protocol. Additionally, the network interface 150 may implement a TCP/IP stack for communication according to the TCP/IP protocols. Various alternative or additional hardware or configurations for the network interface 150 will be apparent.

The network interface 150 may be in operable communication with one or more sensor devices 151. In the healthcare context, these may include sensors configured as part of patient monitoring devices that gather various types of information regarding a patient's health. For example, the one or more sensor devices 151 may include sensors used to conduct a radiology examination.

The type of sensor devices 151 used may of course vary and may depend on the patient, context, and the overall purpose of the examination. Accordingly, any type of sensor devices 151 may be used as long as they can gather or otherwise obtain the required data as part of an examination.

The sensor device(s) 151 may be in communication with the system 100 over one or more networks that may link the various components with various types of network connections. The network(s) may be comprised of, or may interface to, any one or more of the Internet, an intranet, a Personal Area Network (PAN), a Local Area Network (LAN), a Wide Area Network (WAN), a Metropolitan Area Network (MAN), a storage area network (SAN), a frame relay connection, an Advanced Intelligent Network (AIN) connection, a synchronous optical network (SONET) connection, a digital T1, T3, E1, or E3 line, a Digital Data Service (DDS) connection, a Digital Subscriber Line (DSL) connection, an Ethernet connection, an Integrated Services Digital Network (ISDN) line, a dial-up port such as a V.90, a V.34, or a V.34b is analog modem connection, a cable modem, an Asynchronous Transfer Mode (ATM) connection, a Fiber Distributed Data Interface (FDDI) connection, a Copper Distributed Data Interface (CDDI) connection, or an optical/DWDM network.

The network or networks may also comprise, include, or interface to any one or more of a Wireless Application Protocol (WAP) link, a Wi-Fi link, a microwave link, a General Packet Radio Service (GPRS) link, a Global System for Mobile Communication G(SM) link, a Code Division Multiple Access (CDMA) link, or a Time Division Multiple access (TDMA) link such as a cellular phone channel, a Global Positioning System (GPS) link, a cellular digital packet data (CDPD) link, a Research in Motion, Limited (RIM) duplex paging type device, a Bluetooth radio link, or an IEEE 802.11-based link.

The storage 160 may include one or more machine-readable storage media such as read-only memory (ROM), random-access memory (RAM), magnetic disk storage media, optical storage media, flash-memory devices, or similar storage media. In various embodiments, the storage 160 may store instructions for execution by the processor 120 or data upon which the processor 120 may operate.

For example, the storage 160 may include examination data extraction instructions 161, semantic data extraction instructions 162, discrepancy identification instructions 163, and resolution instructions 164. The storage 160 may further include or otherwise have access to one or more ontologies 165 and guidelines established by the American College of Radiology (for simplicity, “ACR guidelines”) 166. Although not specifically shown in FIG. 1, the system 100 may include any appropriate services API to integrate with existing reporting tools and software systems.

The examination data extraction instructions 161 may include rules 167 and natural language processing (NLP) instructions 168 to automatically identify and extract clinical data from an examination report. Specifically, the examination data extraction instructions 161 may include supervised and/or unsupervised machine learning and deep learning rules to extract entities of interest from an examination report. The extracted entities may be based on one or more ontologies 165, word2vec models, regular expressions, or the like, as well as the ACR guidelines 166.

For example, radiology examination findings can be identified and labelled in sentences by keyword matching using one or more of predefined dictionaries or existing ontologies. These ontologies may include, but are not limited to, SNOMED CT® and the Unified Medical Language System (UMLS).

Additionally or alternatively, the examination data can be identified and labelled in sentences by using regular expressions to match a pattern or by using previously labelled data to train any type of appropriate machine learning model. The trained machine learning model(s) may include, but are not limited to, support vector machines, random forests, recurrent neural networks, convolutional neural networks, or any other model that can identify classify findings from the report.

In accordance with various embodiments, the examination data extraction instructions 161 may rely on an ensemble of the approaches described above. For example, the processor 120 may first run keyword matching to identify examination findings. To identify more challenging cases that are rare and not in previously-defined dictionaries or ontologies (e.g., abbreviations that are not widely known), the processor 120 may train a machine learning model based on examples of such cases in order to identify remaining findings that are not detected using keyword matching approaches.

The type of extracted data may vary and may depend on the type of report from which the data is extracted. The extracted data may relate to examination findings, anatomies, diseases, measurements, staging identifications, or the like. In the healthcare context, the type of data extracted from the examination report may of course depend on the type of examination conducted.

For example, the ACR guidelines 166 may cause the processor 120 to extract the required data from the patient's record such as, but not necessarily limited to, nodule size and shape. This data may be extracted directly from an image using image processing techniques (e.g., segmentation) or from a patient's radiology report by executing the NLP instructions 168. As another example, longitudinal data can be extracted from the patient history record to determine the required longitudinal information such as nodule growth over some period of time.

In the event the required features are not available, a clinician or the processor 120 may insert a plurality of ranges (e.g., all possible ranges) of a value for the missing values of the desired data to derive potential ranges of possibilities of outcome. For example, the type of nodule is a feature required by the Fleischner Guidelines. The type of nodule may be classified as ground-glass, sub-solid, or part-solid. If this information is not available, one can derive the suggested data for all three different types of values: Guideline_ground_glass, Guideline_part_solid, and Guideline_sub_solid.

Referring back to FIG. 1, the semantic data extraction instructions 162 may include rules 169 involving supervised and unsupervised machine learning and deep learning components to perform NLP-related tasks. In some embodiments, the one or more ontologies 165 may store semantic relations from a knowledge base that represents expert knowledge in the semantic space, as well as semantic relations created to exploit distributional similarity metrics that represent semantic relations.

The semantic data extraction instructions 162 may execute rules 169 to execute named entity recognition (NER) instructions 170, text entailment instructions 171, anaphora resolution instructions 172, or the like. The processor 120 may execute these instructions with reference to one or more ontologies 165 as well as guidelines such as the ACR guidelines 166.

The NER instructions 170 may enable the processor 120 to recognize or otherwise detect the meaning of certain phrases or words. For example, the NER instructions 170 may recognize the semantic meaning of identified anatomical phrases, diseases, morphological abnormalities, etc.

The text entailment instructions 171 may enable the processor 120 to recognize the relationship between two or more phrases or terms in a report. Specifically, the text entailment instructions 171 may enable the processor 120 to infer semantic meanings of and relationships between terms in the examination report, as well as make inferences regarding data that is not in the examination report.

The anaphora resolution instructions 172 may enable the processor 120 to resolve any anaphoric terms extracted from the examination report. “Anaphors” refer to words or phrases that refer to other words or phrases or relationships in the report. Accordingly, the anaphora resolution instructions 172 may enable the processor 120 to infer relationships, for example, between adjectives and what the adjectives are describing. The anaphora resolution instructions 172 may also leverage one or more ontologies 165 and the ACR 166 for recognizing the relationships between certain words or phrases.

The discrepancy identification instructions 163 may enable the processor 120 to detect discrepancies between the extracted examination data and the extracted semantic data. The discrepancy identification instructions 163 may also rely on one or more ontologies 165 and the ACR guidelines 166 to identify the discrepancies.

For example, in operation, “A” and “B” may represent codified entries representing extracted examination data and extracted semantic data, respectively. The discrepancy identification instructions 163 may check if there is a direct relationship between codified entities “A” and “B” in an ontology 165. If there is no relationship, the processor 120 may flag this instance for correction. For example, the user interface 140 may issue an alert to a user such as a clinician to inform the clinician of the discrepancy.

In some embodiments, a user may receive alerts in real time as they are populating an examination report with notes. That is, a user may input a note regarding a certain finding using any one or more of the previously-discussed techniques. In at least substantially real time (i.e., limited only by processing constraints), the processor 120 may execute the various instructions of storage 160 and issue alerts to a user upon identifying discrepancies.

Some users, however, may not want to be constantly alerted about discrepancies while populating or otherwise completing an examination report. Accordingly, in some embodiments, the user interface 140 may inform a user of all identified discrepancies only after the user has indicated they are finished writing the examination report.

The above embodiments are largely described as rules-based. However, in other embodiments the check for discrepancies may be purely data-driven or a combination or rules-based and data-driven approaches. For example, the systems and methods described herein may involve a training stage based on a large corpus of examination reports. These may involve supervised machine learning procedures to identify relationships between items in examination reports.

In another embodiment, the discrepancy identification instructions 163 may be based on unsupervised or semi-supervised approaches such as adversarial neural networks based on a large corpus of examination reports. In these embodiments, these networks are capable of learning rules on their own and without having access to manually labeled data.

Referring back to FIG. 1, the processor 120 may then execute the resolution instructions 164 to resolve any identified discrepancies. Execution of the resolution instructions 164 may involve receiving input from the user regarding how the user wishes to resolve any identified discrepancies. Alternatively, the processor 120 may execute the resolution instructions 164 to autonomously resolve any identified discrepancies.

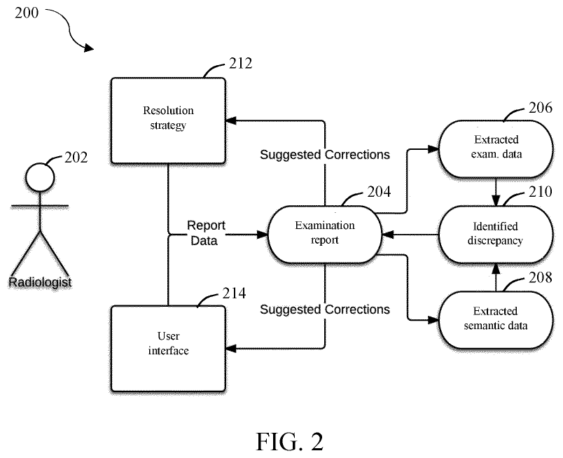

FIG. 2 depicts a workflow 200 of the various components and data of FIG. 1 in accordance with one embodiment. As seen in FIG. 2, a user 202 such as a radiologist may input report data (e.g., related to a patient examination), into an examination report 204.

A processor such as the processor 120 of FIG. 1 may extract examination data 206 and semantic data 208 from the examination report 204. The processor may then identify one or more discrepancies 210 between the extracted examination data 206 and the extracted semantic data 208.

Based on the identified discrepancy 210 and the examination report 204, the processor may suggest one or more corrections to resolve the identified discrepancy. The suggested corrections may be part of an overall resolution strategy 212 regarding how to resolve any identified discrepancies 210. Additionally or alternatively, the suggested corrections may be presented to the user 202 via a user interface 214 such as the user interface 140 of FIG. 1.

The user 202 may then, for example, decide whether to accept or decline the suggested corrections. Similarly, the user 202 may provide input regarding how to resolve the identified discrepancy 210.

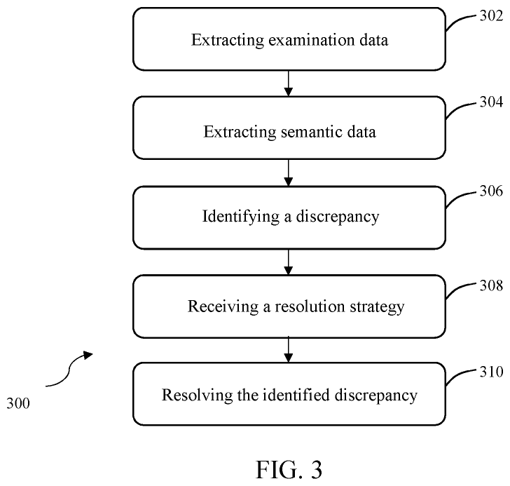

FIG. 3 depicts a flowchart of a method 300 for correcting an examination report in accordance with one embodiment. Method 300 may rely on, e.g., the components of the system 100 of FIG. 1.

Step 302 involves extracting examination data from an examination report. In some embodiments, the examination report may relate to a radiology examination of a patient. The processor 120 of FIG. 1 may perform step 302 by executing the examination data extraction instructions 161. For example, in some embodiments, the processor 120 may rely on word2vec models trained on a corpus of annotated radiology reports. These may include hand-curated examples of clinical language from reports, annotated corpora to train supervised approaches, and larger unlabeled corpora for unsupervised approaches.

The extracted examination data may relate to findings of a patient examination. For example, the extracted examination data may include numerical values or ranges related to some measured health-related parameter. The findings may have been originally entered into the report by a user such as a clinician performing an examination of a patient.

Step 304 involves extracting semantic data from the examination report. The processor 120 of FIG. 1 may perform step 304 by executing the semantic data extraction instructions 162. As discussed previously, the semantic data extraction instructions 162 may enable the processor 120 to use semantic relations from a knowledge base that represents expert knowledge in the semantic space, as well as semantic relations to exploit distributional similarity metrics that represent semantic relations.

Step 306 involves identifying a discrepancy between the extracted examination data and the extracted semantic data. The processor 120 of FIG. 1 may perform this step by executing the discrepancy identification instructions 163. The discrepancy identification instructions 163 may enable the processor 120 to consider the output from steps 302 and 304 and determine whether there are any discrepancies between the extracted examination data and the extracted semantic data.

To detect these discrepancies, the processor 120 may consider relationships in one or more existing ontologies such as SNOMED CT in combination with codes for concepts. For example, each concept has in SNOMED CT has a unique numeric concept identifier known as its “concept id” or its “code.” Accordingly, the processor 120 may consider a concept based on a detected code, and whether there is a discrepancy between the concept and the extracted examination data.

Step 308 involves receiving a resolution strategy regarding how to resolve the identified discrepancy between the extracted examination data and the extracted semantic data. Upon identifying a discrepancy between the extracted examination data and the extracted semantic data, the processor may flag the discrepancy and communicate an alert to a user such as a clinician.

For example, a user interface such as the user interface 140 of FIG. 1 may communicate a visual alert, audio alert, text alert, a haptic-based alert, or some combination thereof to inform the clinician of the identified discrepancy. As discussed previously, these alerts may be communicated to the clinician in real time as the clinician is populating the report, or after the clinician has indicated they are finished with populating the report.

Regardless of when/how the alerts regarding any identified discrepancies are communicated to the user, the user may then provide input regarding how to resolve the identified discrepancy. For example, the user may specify to which anatomical part they are referring in a report. The exact input provided (i.e., the resolution strategy provided) may vary and may depend on the identified discrepancy.

In some embodiments, the processor 120 may execute the resolution instructions 164 to obtain an appropriate resolution strategy. For example, the processor 120 may consider data from one or more ontologies 165, guidelines such as the ACR 166, and previously-generated reports to autonomously develop a resolution strategy.

Step 310 involves resolving the identified discrepancy between the extracted examination data and the extracted semantic data based on the resolution strategy. The processor 120 may then execute the received resolution strategy, whether provided by a user or generated autonomously. The “corrected” examination report may then be stored in a database or presented to a user.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, or alternatively, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Having described several example configurations, various modifications, alternative constructions, and equivalents may be used without departing from the spirit of the disclosure. For example, the above elements may be components of a larger system, wherein other rules may take precedence over or otherwise modify the application of various implementations or techniques of the present disclosure. Also, a number of steps may be undertaken before, during, or after the above elements are considered.

Having been provided with the description and illustration of the present application, one skilled in the art may envision variations, modifications, and alternate embodiments falling within the general inventive concept discussed in this application that do not depart from the scope of the following claims.

1. A computer implemented method for correcting a medical examination report, the method comprising:
extracting examination data from the examination report, the examination data relating to findings of an examination;
extracting semantic data from the examination report, the semantic data relating to semantic meanings of linguistic structures in the examination report;
identifying a discrepancy between the extracted examination data and the extracted semantic data using one or more ontologies;
receiving a resolution strategy regarding how to resolve the identified discrepancy between the extracted examination data and the extracted semantic data; and
resolving the identified discrepancy between the extracted examination data and the extracted semantic data based on the resolution strategy.

2. The method of claim 1 wherein the examination report relates to a radiology examination.

3. The method of claim 1 further comprising presenting, using a user interface, the identified discrepancy to a user, wherein receiving the resolution strategy includes receiving, using the user interface, user feedback regarding how to resolve the identified discrepancy between the extracted examination data and the extracted semantic data.

4. The method of claim 1 wherein a user provides the examination data to the examination report and includes at least one of anatomies, diseases, measurements, and staging identifications related to an examination.

5. The method of claim 1 wherein identifying the discrepancy comprises consulting an ontology to determine whether a relationship exists between the extracted examination data and the extracted semantic data, wherein the discrepancy is identified upon determining that a relationship does not exist between the extracted examination data and the extracted semantic data.

6. The method of claim 1 wherein identifying the discrepancy comprises identifying the discrepancy using a neural network machine learning model trained using training examination data and training semantic data to identify relationships between extracted examination data and extracted semantic data.

7. (canceled)

8. A system for correcting a medical examination report, the system comprising:
an interface for receiving an examination report;
a processor executing instructions stored on a memory to:
extract examination data from the examination report, the examination data relating to findings of an examination,
extract semantic data from the examination report, the semantic data relating to semantic meanings of linguistic structures in the examination report,
identify a discrepancy between the extracted examination data and the extracted semantic data using one or more ontologies,
receive a resolution strategy regarding how to resolve the identified discrepancy between the extracted examination data and the extracted semantic data, and
resolve the identified discrepancy between the extracted examination data and the extracted semantic data based on the resolution strategy.

9. The system of claim 8 wherein the examination report relates to a radiology examination.

10. The system of claim 8 further comprising a user interface for:
presenting the identified discrepancy to a user, and
receiving the resolution strategy from the user.

11. The system of claim 8 wherein the examination data includes at least one of anatomies, diseases, measurements, and staging identifications related to an examination.

12. The system of claim 8 wherein the processor is further configured to identify the discrepancy by consulting an ontology to determine whether a relationship exists between the extracted examination data and the extracted semantic data, wherein the discrepancy is identified upon the processor determining that a relationship does not exist between the extracted examination data and the extracted semantic data.

13. The system of claim 12 wherein the processor is further configured to identify the discrepancy using a neural network machine learning model using training examination data and training semantic data to identify relationships between extracted examination data and extracted semantic data.

14. (canceled)

15. A non-transitory computer-readable medium containing computer executable instructions for performing a method for correcting a medical examination report, the computer-readable medium comprising:
computer-executable instructions for extracting examination data from the examination report, the examination data relating to findings of an examination;
computer-executable instructions for extracting semantic data from the examination report, the semantic data relating to semantic meanings of linguistic structures in the examination report;
computer-executable instructions for identifying a discrepancy between the extracted examination data and the extracted semantic data, using one or more ontologies;
computer-executable instructions for receiving a resolution strategy regarding how to resolve the identified discrepancy between the extracted examination data and the extracted semantic data; and
computer-executable instructions for resolving the identified discrepancy between the extracted examination data and the extracted semantic data based on the resolution strategy.
